(19) United States
(12) Patent Application Publication
(10) Pub. No.: US 2004/0229342 A1
(43) Pub. Date: Nov. 18, 2004

(54) Process for remediation of hydrocarbons

(76) Inventors: Ken Lunde, La Quinta, CA (US); John Zaabadick, Milltown, NJ (US)

Correspondence Address: FULBRIGHT AND JAWORSKI L L P, PATENT DOCKETING 29TH FLOOR, 865 SOUTH FIGUEROA STREET, LOS ANGELES, CA, 900172576

(21) Appl. No.: 10/440,029

(22) Filed: May 16, 2003

(51) Int. Cl. C12P 21/02

(52) U.S. Cl. 435/262

(57) ABSTRACT

A process for the remediation and conditioning of soil containing a contaminant. The process comprises (a) mixing the soil with solid material comprising a source of microbial nourishment, (b) inoculating the mixed soil with a diverse microbe population capable of digesting the contaminant, and (c) maintaining the moisture content of the inoculated soil at a level sufficient to support microbial growth. The process can include the additional steps of applying a suspension fertilizer to the mixed soil and/or applying an oxygen-releasing compound to the mixed soil. Step (b) can be repeated to sufficiently reduce the level of contamination.

### BACKGROUND OF THE INVENTION

This invention relates generally to soil remediation and specifically to the bioremediation of contaminated soil.

### RELATED ART

The production, transportation and storage of hazardous chemicals including petroleum products has led to environmental contamination with resulting ecosystem damage. The harm caused by soil contamination in particular includes the accumulation of contaminants in plants and animals, and the contamination of local groundwater supplies.

Generally, two approaches are available for the remediation of contaminated soil. In one approach, contaminants are removed from soil by physical means such as heating, incineration, or vacuum extraction. However, the excavation of soil required for heating and incineration is expensive. Further, vacuum extraction does not effectively remove many pollutants, for example those with high vapor pressures. In the second approach, excavated contaminated soil is spread out across a ground surface and kept moist to allow resident or added microbes to degrade the contaminant (a process called “farming” in the industry). However, such bioremediation has failed to reduce hydrocarbon contaminants, or has been a slow process only gradually reducing levels of contamination over a relatively long period of time.

### SUMMARY OF THE INVENTION

The present invention provides a remediation process for contaminated soil. Relative to physical remediation, the process is simple and economical to employ. The invention is particularly suitable for the remediation of soil containing petroleum and hydrocarbon contaminants. Although the process is designed to remediate soil containing a contaminant, the process can simultaneously nurture and condition the soil.

The process of the present invention comprises (a) mixing contaminated soil with solid material including at least one source of microbial nourishment, (b) inoculating the mixed soil with a diverse microbe population capable of digesting the contaminant, and (c) maintaining the moisture content of the inoculated soil at a level sufficient to support microbial growth. In particular versions of the invention, the process can include the additional steps of applying a suspension fertilizer to the mixed soil and/or applying an oxygen-releasing compound to the mixed soil.

The novel features which are believed to be characteristic of the invention together with further objects and advantages will be better understood from the following description and examples. It is to be expressly understood, however, that each example is provided for the purpose of illustration and description only and is not intended to define the limits of the present invention.

### DETAILED DESCRIPTION OF THE PREFERRED EMBODIMENTS

For soil remediation, at least one source of microbial nourishment is mixed into the target soil. Any microbial food source can be utilized. Preferred sources of nourishment include manure, compost, plant materials and carbohydrate. Although the manure can be from any animal such as cow, sheep, goat, horse, chicken and pig, the manure is preferably cow or sheep manure, with cow manure being preferred. The manure should be sufficiently aged to prevent toxic effects on the soil. Manure aged about one to three years is suitable. Preferably, the manure is aged about two years and is finely granulated. However, manure of any size that adequately supports microbial growth in the soil can be utilized.

The compost can be any compost, preferably a rich compost near the humus stage. Methods of preparing compost are well known in the art of composting.

The plant material can be any cellulose material that is digestible by microbes, including such materials as cotton trash, hay, grasses, milled crop materials, and compost-type materials well known in the art. A preferred plant material is cotton trash, which is often used as a food supplement for livestock. The granular size of the plant material can be about ½ inch to facilitate breakdown in the soil, with sizes smaller than about ½ inch being preferred.

The carbohydrate can be simple or complex, or a mixture of the two. Suitable carbohydrates include sucrose, glucose, maltose, dextrose, fructose, and the like. Preferably, the carbohydrate is sucrose, which can be conveniently added in the form of brown sugar.

The source of microbial nourishment can be a mixture of manure and plant material, or a mixture of manure, plant material and carbohydrate. Such mixtures can provide a diverse and rich food supply for supporting microbial growth. Suitable proportions for each component can be in the range of about 70% to 95% manure, 5% to 25% plant material, and 2% to 10% carbohydrate, by weight. Preferable ranges are about 80% to 90% manure, 5% to 18% plant material, and 2% to 5% carbohydrate, by weight. When the mixture comprises all three components, a preferable mix of components is about 90% manure, 8% plant material, and 2% carbohydrate, by weight. As will be apparent to those skilled in the art, other proportions are possible as long as the final mixture effectively supports soil remediation.

The contaminated soil is inoculated with a diverse microbe population capable of digesting the contaminant. As used herein, a diverse microbe population contains bacteria and fungi of more than one genus and species. Preferably, the microbe population contains at least 10² different microorganisms. Sources of such diverse microbe populations are well known in the art and can be sources utilized in bioremediation processes such as composting, landfarming, slurry biodegradation, and heap pile bioremediation. Preferably, the microbe population comprises naturally occurring microorganisms. Exemplary microorganisms that can be included in a diverse microbe population include Candida lipolytica, Candida tropicalis, Pseudomonas methanica, Pseudomonas methanitrificans, Methylococcus capsulatus, Aerobacter, aerogenes, Pseudomonas aeruginosa, Pseudomonas putida, Pseudomonas alcaligenes, Bacillus megaterium, Bacillus licheniformis, various Nocardia species, other Candida species, various Streptomyces species, various yeasts and various fungi.

The soil can be inoculated by methods well known in the art such as mixing, spreading or injection into the soil. A particularly desirable method of inoculation is to spray the soil with a diverse microbe population suspended in an aqueous carrier. Spraying of the microbe population provides for simultaneous inoculation and wetting of the soil.

The moisture level of the soil is preferably maintained at a moisture content of about 20% to 50% by weight, and more preferably about 30%, to support microbe growth in the treated soil. Depending on the temperature, wind, occurrence of rain and other environmental conditions, the treated soil can be re-wetted by spraying with water to maintain the moisture level.

The process can include the step of applying a suspension fertilizer to the mixed soil. The suspension fertilizer can be applied by well known methods such as mixing, spreading, broadcasting, and preferably spraying. As used herein, a suspension fertilizer contains colloidal particles of an organic material, as exemplified in U.S. Pat. No. 5,443,613 to Robinson, herein incorporated by reference.

It is well known that oxygen releasing compounds, particularly hydrogen peroxide, can increase the oxygen content of soil, thus improving bioremediation. Therefore, as an additional step in the remediation process, an oxygen-releasing compound can be applied to the mixed soil by well known methods such as mixing, spreading, broadcasting, and preferably spraying. A particularly suitable oxygen releasing compound for application by spraying is hydrogen peroxide, which can be diluted to a level that is safe both to the person spraying the solution and to the microbes in the treated soil.

In practice, manure, plant material and carbohydrate can be mixed with the aid of an agricultural grinder or mixer. The blended mix can be applied to the soil at a rate of about one pound per square foot of surface area, with the mix spread out evenly on the surface of the contaminated soil. Small surface areas can be easily spread by hand with a rake. Larger areas can be conveniently prepared by use of equipment such as a backhoe, an agricultural spreader, or the like. The solid material can be thoroughly mixed into the contaminated soil surface using a tiller, a small plow, a disk, or the like. A four to seven foot tiller is effective at mixing, aerating, and further breaking down the size of the contaminated soil. A garden size tiller can be used in and around tanks, flow lines and other infrastructure where space is limited.

If the contaminated soil is more than a foot deep, the site is preferably prepared by digging out the contaminated soil and placing it in a pile for mixing with a backhoe or loader. This ensures that the contaminant is evenly dispersed throughout the soil. The soil can then be evenly spread out to a depth of not more than about twelve inches in preparation for mixing with the solid material. A depth of nine to ten inches is preferred, allowing for optimum penetration of oxygen and facilitating mixing with the solid material. Contaminated soil that is twelve inches or less deep can be remediated in situ without digging out.

Compaction of the soil with heavy equipment should be avoided. Also, large rocks and other foreign debris should be removed.

A suitable preparation of a diverse microbe population is VESTA® (Biologically Integrated Organics, Inc., Fresno, Calif.), a commercially available microbe preparation in liquid form containing at least 187 different microorganisms. The commercial microbe preparation can be applied at a rate of about three to five ounces per thousand square feet of surface area. The three to five ounces can be suspended in about 100 gallons of water, and the suspension sprayed onto the mixed soil.

A suitable commercial suspension fertilizer, which is prepared in accordance with U.S. Pat. No. 5,443,613, is available from The Dune Company (El Centro, Calif.). The fertilizer can be applied at a rate of about one and three quarters gallons of the commercial suspension fertilizer per thousand square feet of surface area. The commercial fertilizer can be diluted with about one hundred gallons of water for spraying onto the mixed soil. Although the suspension fertilizer and the microbial population can be mixed and applied together, it is preferable to apply the components in separate steps to minimize microbe mortality.

Dilute hydrogen peroxide can be prepared by dissolving about one gallon of commercial grade (30%) hydrogen peroxide in about three hundred gallons of water for spraying over a thousand square feet of surface area.

To apply the liquid components, a four hundred gallon water tank can be connected to a centrifuge pump having a discharge capacity of one hundred and forty gallons per minute. A firehose nozzle and a two inch diameter discharge hose can be employed to apply the liquid. The pressure of the liquid should be controlled during application to avoid damaging the surface of the remediation site. Additionally, the liquid components should be sprayed in such a manner that disruption of the soil surface and loss of applied materials are minimized. It is preferable to apply the liquid components during cool and calm times of the day.

Following application of the liquid components, the treated soil is preferably left undisturbed.

In some applications, the step of spraying the mixed soil with a diverse microbe population suspended in an aqueous carrier can be repeated one or more times to further reduce the level of contamination to desired levels. For repeat applications, about one to three ounces of the commercial microbe preparation, in about one hundred gallons of water, can be applied per thousand square feet of surface area. Intervals of five to seven days between repeated applications are preferred. Before re-applying the microbe population, the soil can be aerated by turning it over with a tiller, small plow, disk, or the like.

As will be obvious to those skilled in the art, the amounts and proportions of the various components of the present invention can be varied to accelerate the remediation process, and to accommodate different environmental conditions and different levels of contamination.

In general, the remediation process is preferably carried out when the ground temperature is about 55° F. or higher, and preferably not below about 45° F. Also, wind speeds are preferably less than 30 mph to prevent uneven application of liquid material.

Without wishing to be limited by any theory, two factors appear important. First, a rich source of food for the microbial population allows robust growth in the soil. A multiplicity of food sources such as a mixture of manure, plant material and carbohydrate is a particularly rich source of microbial nourishment. Second, by inoculating the soil with a diverse microbe population, the process provides a multiplicity of different types of microorganisms that work together to break down contaminants and restore the soil to a healthy state. The application of manure, plant material and suspension fertilizer, which are additional sources of microbes, can enhance the diversity of microbes added to the soil. The process can be further understood by referring to the following examples.

### EXAMPLES

### Example 1

A contamination site of about 1200 cubic feet of soil in a cotton field bordering an active oil well was treated. The soil was farmland and easy to work, and the depth of contamination was such that digging out of the soil was not required. The remediation process was carried out using a mixture of about 90% cow manure, 8% cotton trash, and 2% sucrose as the source of microbial nourishment. The soil was sprayed with a suspension of the commercial microbe preparation (Biologically Integrated Organics, Inc., Fresno, Calif.), a solution of the commercial suspension fertilizer (The Dune Company), and dilute hydrogen peroxide, all prepared as described herein. Only a single application of the microbe population was necessary.

The level of contamination before treatment was about 1.800% total parts hydrocarbon (“tph”). Contamination levels of representative soil samples were determined by independent laboratories employing EPA approved testing methods, typically the EPA 418.1 (freon extraction) method. A level of 1.000% tph or less was considered indicative of complete treatment. In this example, the climatic conditions were considered severe, with drought and strong winds.

After 35 days, the level of contamination was reduced to about 1.000% tph.

### Example 2

A contamination site of about 1200 cubic feet of soil in an oil storage battery was treated. The site was treated in situ since the depth of contamination was about one foot. The soil was desert sand containing old and new crude oil contamination. The area was extremely windy and about ¾ of an inch of rain was deposited on the site during treatment. The remediation process was carried out as in Example 1 with two additional applications of the commercial microbe preparation. Microbes were first reapplied seven days after the initial application. Microbes were reapplied a second time about two weeks following the first reapplication.

The initial level of contamination was about 4.9000% tph. After 45 days, the level of contamination was reduced to about 0.2416% tph.

### Example 3

The contamination site was about 5,000 cubic feet of soil around an abandoned oil well. The soil was limestone based. The remediation process was carried out in situ as in Example 1 with one additional application of microbes performed ten days following initial treatment.

The initial level of contamination was 1.6000% tph. After 45 days, the level of contamination was reduced to less than 1.0000% tph.

### Example 4

About 6000 cubic feet of contaminated soil from a cotton field around an oil flow line was treated. Using a backhoe, the soil was dug out and mixed to evenly disperse the contamination and to aerate the soil. Solid material containing 90% cow manure, 8% cotton trash, and 2% sucrose was then evenly mixed into the soil, and the soil was spread out to a depth of about one foot. Next, the soil was sprayed with a suspension of the commercial microbe preparation, a solution of the commercial suspension fertilizer, and dilute hydrogen peroxide, as in Example 1. The microbe population was reapplied two weeks after the initial treatment, following some rainfall.

The initial level of contamination was about 3.6576% tph. After 55 days, the level of contamination was reduced to about 1.0090% tph.

### Example 5

About 2400 cubic feet of contaminated soil in two locations of an oil storage battery was treated. The soil was limestone based, and the conditions were hot, dry and windy. The remediation process was carried out by altering the composition of microbes, suspension fertilizer, and manure. Compared to Example 1, the amount of the commercial microbe preparation used per thousand square feet of surface area was doubled, the amount of the commercial suspension fertilizer was increased by 50%, and the amount of manure was increased to two pounds per square feet of surface area. The amounts of cotton trash, hydrogen peroxide and sucrose remained the same as in Example 1.

The initial level of contamination at the first location was about 6.6129% tph, and at the second location, about 5.6207% tph. After 40 days, the level of contamination at the first location was reduced to about 0.1097% tph, and at the second location, to about 0.6729% tph.

### Example 6

About 4500 cubic feet of contaminated soil from an oil storage battery was dug out, evenly mixed, then spread out to a depth of one foot. The soil was treated essentially as in Example 3, but with the commercial microbe preparation, commercial suspension fertilizer, and manure one and a half times more concentrated. Two additional applications of the commercial microbe preparation, one at five days following initial treatment, the other at two weeks after the first reapplication of microbes, were performed. The weather was extremely dry with calm winds.

The initial level of contamination was about 2.6700% tph. After 30 days, the level of contamination was reduced to about 0.6000% tph. An interim test performed at seven days following initial treatment showed a tph level of only about 1.4000%.

What is claimed is

1. A process for the remediation and conditioning of soil containing a contaminant, comprising the steps of:
(a) mixing the soil with solid material comprising a source of microbial nourishment;
(b) inoculating the mixed soil with a diverse microbe population capable of digesting the contaminant; and
(c) maintaining the moisture content of the inoculated soil at a level sufficient to support microbial growth.

2. The process of claim 1 wherein the source of microbial nourishment is manure, compost, plant material, or carbohydrate.

3. The process of claim 2 wherein the source of microbial nourishment is manure.

4. The process of claim 3 wherein the manure is cow, sheep, goat, horse, chicken or pig manure.

5. The process of claim 4 wherein the manure is cow manure.

6. The process of claim 2 wherein the source of microbial nourishment is compost.

7. The process of claim 2 wherein the source of microbial nourishment is plant material.

8. The process of claim 7 wherein the plant material is cotton trash, hay, grasses, milled crop materials, or compost-type materials.

9. The process of claim 8 wherein the plant material is cotton trash.

10. The process of claim 2 wherein the carbohydrate is a complex carbohydrate.

11. The process of claim 2 wherein the carbohydrate is a simple carbohydrate.

12. The process of claim 11 wherein the simple carbohydrate is sucrose, glucose, maltose, dextrose, or fructose.

13. The process of claim 12 wherein the simple carbohydrate is sucrose.

14. The process of claim 2 wherein the carbohydrate is a mixture of simple and complex carbohydrate.

15. The process of claim 1 wherein the source of microbial nourishment is a mixture of manure and plant material.

16. The process of claim 1 wherein the source of microbial nourishment is a mixture of manure, plant material and carbohydrate.

17. The process of claim 16 wherein the mixture is about 90% manure, 8% plant material, and 2% carbohydrate, by weight.

18. The process of claim 16 wherein the manure is cow manure, the plant material is cotton trash, and the carbohydrate is sucrose.

19. The process of claim 18 wherein the mixture is about 90% cow manure, 8% cotton trash, and 2% sucrose, by weight.

20. The process of claim 1 wherein the microbe population is applied to the mixed soil by spraying.

21. The process of claim 1 wherein the microbe population comprises naturally occurring microorganisms.

22. The process of claim 1 wherein the microbe population comprises at least 10² different microorganisms.

23. The process of claim 1 wherein the microbe population is a hydrocarbon digesting population.

24. The process of claim 1 further comprising the step of applying a suspension fertilizer to the mixed soil.

25. The process of claim 24 wherein the suspension fertilizer is applied to the mixed soil by spraying.

26. The process of claim 24 further comprising the step of applying an oxygen releasing compound onto the mixed soil.

27. The process of claim 26 wherein the oxygen releasing compound is applied to the mixed soil by spraying.

28. The process of claim 26 wherein the oxygen releasing compound is hydrogen peroxide.

29. The process of claim 1 further comprising the step of applying an oxygen releasing compound onto the mixed soil.

30. The process of claim 29 wherein the oxygen releasing compound is applied to the mixed soil by spraying.

31. The process of claim 29 wherein the oxygen releasing compound is hydrogen peroxide.

32. The process of claim 1 further comprising repeating step (b) a sufficient number of times to reduce the level of contamination.

33. A process for the remediation and conditioning of soil containing a contaminant, comprising the steps of:
(a) mixing the soil with solid material comprising a mixture of manure, plant material and carbohydrate;
(b) inoculating the mixed soil with a diverse microbe population capable of digesting the contaminant;
(c) spraying a suspension fertilizer onto the mixed soil; and
(d) maintaining the moisture content of the inoculated soil at a level sufficient to support microbial growth.

34. The process of claim 33 wherein the mixture is about 90% manure, 8% plant material, and 2% carbohydrate, by weight.

35. The process of claim 33 wherein the manure is cow manure.

36. The process of claim 33 wherein the plant material is cotton trash.

37. The process of claim 33 wherein the carbohydrate is sucrose.

38. The process of claim 33 wherein the microbe population is applied to the mixed soil by spraying.

39. The process of claim 33 wherein the microbe population comprises naturally occurring microorganisms.

40. The process of claim 33 wherein the microbe population is a hydrocarbon digesting population.

41. The process of claim 33 wherein the microbe population comprises at least 10² different microorganisms.

42. The process of claim 33 further comprising the step of applying an oxygen releasing compound onto the mixed soil.

43. The process of claim 42 wherein the oxygen releasing compound is applied to the mixed soil by spraying.

44. The process of claim 42 wherein the oxygen releasing compound is hydrogen peroxide.

45. The process of claim 33 further comprising repeating step (b) a sufficient number of times to reduce the level of contamination.

46. A process for the remediation and conditioning of soil containing a contaminant, comprising the steps of:
(a) mixing the soil with solid material comprising a mixture of manure, plant material and carbohydrate;
(b) inoculating the mixed soil with a diverse microbe population capable of digesting the contaminant;
(c) spraying a suspension fertilizer onto the mixed soil;
(d) spraying hydrogen peroxide onto the mixed soil; and
(e) maintaining the moisture content of the inoculated soil at a level sufficient to support microbial growth.

47. The process of claim 46 wherein the mixture is about 90% manure, 8% plant material, and 2% carbohydrate, by weight.

48. The process of claim 46 wherein the manure is cow manure.

49. The process of claim 46 wherein the plant material is cotton trash.

50. The process of claim 46 wherein the carbohydrate is sucrose.

51. The process of claim 46 wherein the microbe population is applied to the mixed soil by spraying.

52. The process of claim 46 wherein the microbe population comprises naturally occurring microorganisms.

53. The process of claim 46 wherein the microbe population is a hydrocarbon digesting population.

54. The process of claim 46 wherein the microbe population comprises at least 10² different microorganisms.

55. The process of claim 46 further comprising repeating step (b) a sufficient number of times to reduce the level of contamination.

56. A process for the remediation and conditioning of soil containing a hydrocarbon contaminant, comprising the steps of:
(a) mixing the soil with solid material comprising a mixture of cow manure, cotton trash and sucrose;
(b) spraying the mixed soil with a diverse microbe population capable of digesting the hydrocarbon contaminant;
(c) spraying a suspension fertilizer onto the mixed soil;
(d) spraying hydrogen peroxide onto the mixed soil; and
(e) maintaining the moisture content of the inoculated soil at a level sufficient to support microbial growth.

57. The process of claim 56 wherein the mixture is about 90% cow manure, 8% cotton trash, and 2% sucrose, by weight.

58. The process of claim 56 further comprising repeating step (b) a sufficient number of times to reduce the level of contamination.

59. The process of claim 56 wherein the microbe population comprises naturally occurring microorganisms.

60. The process of claim 56 wherein the microbe population comprises at least 10² different microorganisms.
